(19) United States
(12) Patent Application Publication
(10) Pub. No.: US 2024/0108918 A1
(43) Pub. Date: Apr. 4, 2024

(54) COMPUTER-IMPLEMENTED METHOD AND SYSTEM TO PROVIDE ALTERNATIVE FLAVOR COMPOSITIONS

(71) Applicant: Firmenich SA, Satigny (CH)

(72) Inventors: Igor BODNAR, Satigny (CH); Eugénie CHANEL, Neuilly-Sur-Seine (FR); Alan PARKER, Satigny (CH)

(21) Appl. No.: 18/477,239

(22) Filed: Sep. 28, 2023

(30) Foreign Application Priority Data
Sep. 28, 2022 (EP) 22198324.0

(51) Int. Cl. A61Q 13/00

(52) U.S. Cl. CPC A61Q 13/00

(57) ABSTRACT

The computer-implemented method (100) to provide alternative flavor compositions, comprises:
a step (105) of inputting, upon a computer interface, at least one flavor ingredient digital identifier, the resulting input corresponding to a composition of flavor ingredients, each ingredient digital identifier being associated with at least one equivalent constitutive molecule, each equivalent constitutive molecule being associated with at least one property,
a step (110) of transposing, by a computing system, the input composition into a list of at least one equivalent constitutive molecule as a function of at least one input ingredient digital identifier,
a step (115) of defining, upon a computer interface, at least one reformulation rule to be applied to the at least one transposed equivalent constitutive molecule and/or to at least one alternative flavor ingredient obtained during a step of determining,
the step (120) of determining, by a computing system, a selection of at least one alternative flavor ingredient represented by at least one alternative flavor ingredient digital identifier as a function of at least one reformulation rule, the resulting selection corresponding to an alternative composition of flavor ingredients, and
a step (125) of providing, upon a computer interface, the determined alternative composition of flavor ingredients.

### TECHNICAL FIELD OF THE INVENTION

The present invention aims at a computer-implemented method to provide alternative flavor compositions and a system to provide alternative flavor compositions.

It applies, in particular, to the industry of flavor production.

### BACKGROUND OF THE INVENTION

The field of flavor design and production is a dynamic industry in which ingredients, whether natural or synthetic, evolve in availability, desirability, cost, legal status, impact on the climate and so on.

Such evolutions require regular reformulation of existing flavor compositions, a particular blend of flavor ingredients. Such reformulations are typically time-consuming, prone to errors and may be sub-optimized. In current systems, such reformulations are performed by human beings that rely on empirical knowledge and suffer from ordinary biases.

For these reasons, current approaches are unsatisfactory to reliably reformulate flavor compositions in view of particular reformulation constraints.

### SUMMARY OF THE INVENTION

The present invention aims at addressing all or part of these drawbacks.

The overarching concept of this invention relates to the use of two distinct relationships:
the first relationship equates flavor ingredients to constitutive molecules of said ingredients, meaning that the decomposition of ingredients into molecules is known and digitized into an accessible computer database or memory, and
the second relationship equates ingredients and molecules to properties, be it psychophysical properties, hedonic properties, financial properties, sustainability properties and so on, meaning that the association of molecules to said properties is known and digitized into an accessible computer database or memory.

As flavorists typically do not engage with molecules directly, given the number of molecules and variable associated properties, the use of these two distinct relationships allows for dynamic, real-time composition reformulation. Provided a flavorist is able to specify a list of constraints, or rules, that an intended composition must abide by, a computerized system may then select the optimal ingredients, to obtain the initially selected molecules (induced by the selection of initial ingredients), or their equivalents from a flavoring perspective, and provide the alternative composition of ingredients to the flavorist.

According to a first aspect, the present invention aims at a computer-implemented method to provide alternative flavor compositions, comprising:
a step of inputting, upon a computer interface, at least one flavor ingredient digital identifier, the resulting input corresponding to a composition of flavor ingredients, each ingredient digital identifier being associated with at least one equivalent constitutive molecule, each equivalent constitutive molecule being associated with at least one property,
a step of transposing, by a computing system, the input composition into a list of at least one equivalent constitutive molecule as a function of at least one input ingredient digital identifier,
a step of defining, upon a computer interface, at least one reformulation rule to be applied to the at least one transposed equivalent constitutive molecule and/or to at least one alternative flavor ingredient obtained during a step of determining,
the step of determining, by a computing system, a selection of at least one alternative flavor ingredient represented by at least one alternative flavor ingredient digital identifier as a function of at least one reformulation rule, the resulting selection corresponding to an alternative composition of flavor ingredients, and
a step of providing, upon a computer interface, the determined alternative composition of flavor ingredients.

Such provisions allow for the automatic reformulation of flavor compositions in view of customizable reformulation rules, or constraints. Such automation allows for faster, stable, and more optimized reformulation than the manual reformulation techniques used in current systems.

In particular embodiments, the method object of the present invention comprises:
a step of entering, upon a computer interface, at least one secondary reformulation rule to be applied on the alternative composition of flavor ingredients determined,
a step of optimizing, by a computer system, the alternative composition of flavor ingredients determined as a function of the entered at least one secondary reformulation rule.

Such embodiments allow for the subsequent optimization of a reformulated composition in view of a secondary target, such as cost for example.

In particular embodiments, at least one reformulation rule is representative of a geographical region of origin of at least one ingredient, each ingredient being associated with at least one property representative of a geographical region of origin of said ingredient.

In particular embodiments, at least one reformulation rule is representative of a molecule production ecological impact status of at least one equivalent constitutive molecule, each equivalent constitutive molecule being associated with at least one property representative of a molecule production ecological impact of said equivalent constitutive molecule.

In particular embodiments, at least one reformulation rule is representative of a molecule availability of at least one equivalent constitutive molecule, each equivalent constitutive molecule being associated with at least one property representative of a molecule availability of said equivalent constitutive molecule.

In particular embodiments, at least one reformulation rule is representative of a Henry constant of at least one equivalent constitutive molecule, each equivalent constitutive molecule being associated with at least one property representative of a Henry constant of said equivalent constitutive molecule.

In particular embodiments, at least one reformulation rule is representative of an odor detection threshold of at least one equivalent constitutive molecule, each equivalent constitutive molecule being associated with at least one property representative of an odor detection threshold of said equivalent constitutive molecule.

In particular embodiments, at least one reformulation rule is representative of a dose response curve of at least one equivalent constitutive molecule, each equivalent constitutive molecule being associated with at least one property representative of a dose response curve of said equivalent constitutive molecule.

In particular embodiments, each equivalent constitutive molecule is associated with at least one olfactory property, the step of determining being further performed as a function of at least one said olfactory property.

Such embodiments allow for the consideration, on top of reformulation rules, of olfactory properties of ingredients.

In particular embodiments, the method object of the present invention comprises a step providing, to an assembling device, an assembling command of the alternative composition of flavor ingredients.

Such embodiments allow for the materialization of the reformulated composition.

According to a second aspect, the present invention aims at a system to provide alternative flavor compositions, comprising:
means of inputting, upon a computer interface, at least one flavor ingredient digital identifier, the resulting input corresponding to a composition of flavor ingredients, each ingredient digital identifier being associated with at least one equivalent constitutive molecule, each equivalent constitutive molecule being associated with at least one property,
means of transposing, by a computing system, the input composition into a list of at least one equivalent constitutive molecule as a function of at least one input ingredient digital identifier,
means of defining, upon a computer interface, at least one reformulation rule to be applied to the at least one transposed equivalent constitutive molecule and/or to at least one alternative flavor ingredient obtained by means of determining,
said means of determining, by a computing system, a selection of at least one alternative flavor ingredient represented by at least one alternative flavor ingredient digital identifier as a function of at least one reformulation rule, the resulting selection corresponding to an alternative composition of flavor ingredients, and
means of providing, upon a computer interface, the determined alternative composition of flavor ingredients.

Such provisions provide similar benefits to those of the first aspect of the present invention.

### BRIEF DESCRIPTION OF THE DRAWINGS

Other advantages, purposes and particular characteristics of the invention shall be apparent from the following non-exhaustive description of at least one particular embodiment of the present invention, in relation to the drawings annexed hereto, in which:

FIG. 1 shows, schematically, a particular succession of steps of the method object of the present invention,

FIG. 2 shows, schematically, a particular embodiment of the system object of the present invention,

FIG. 3 shows, schematically, a particular embodiment of a graphic user interface used to embody the method object of the present invention, and

FIG. 4 shows, schematically, an example of the concept at the basis of the present invention.

### DETAILED DESCRIPTION OF THE INVENTION

This description is not exhaustive, as each feature of one embodiment may be combined with any other feature of any other embodiment in an advantageous manner.

Various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

It should be noted at this point that the figures are not to scale.

The terms “composition” or formula designates a liquid, solid or gaseous assembly of at least one volatile ingredient.

As used herein, a “flavor” refers to the olfactory perception resulting from the sum of odorant receptor(s) activation, enhancement, and inhibition (when present) by at least one volatile ingredient via orthonasal and retronasal olfaction as well as activation of the taste buds which contain taste receptor cells. Accordingly, by way of illustration and by no means intending to limit the scope of the present disclosure, a “flavor” results from the olfactory and taste bud perception arising from the sum of a first volatile ingredient that activates an odorant receptor or taste bud associated with a coconut tonality, a second volatile ingredient that activates an odorant receptor or taste bud associated with a celery tonality, and a third volatile ingredient that inhibits an odorant receptor or taste bud associated with a hay tonality.

As used herein, “olfactory tonality” or “tonality” means an organoleptic property of a volatile molecule initiated by the activation of an OR, which activity is routed through the olfactory bulb and processed by the central nervous system to produce a specific experience in the subject. Said experience is of a nature that may be described by the subject linguistically, often with reference to everyday objects with which the olfactory experience is associated. Additional modifiers are typically required to capture the specificity of the tonality. Everyday objects that possess a characteristic “smell” typically elicit the experience of multiple separable tonalities when evaluated by a trained expert such as a perfumer. The experience of a single tonality is entirely dependent on the specific OR activated in the subject, such that a single OR produces the perception of a single tonality and the multitude of tonalities produced by may everyday objects is determined by the set of ORs activated by the set of volatile molecules they emit. Though the mechanism differs in many important ways, the perception of an olfactory tonality may be considered analogous to the perception of a single discernable musical note through the auditory system, discernable even when multiple notes are played at once. Similarly, where the same musical note may be described in multiple ways (F-sharp, G-flat, etc.), so the same olfactory tonality may be described in multiple ways.

Non-limiting examples of an olfactory tonality include earthy, coconut, celery, hay etc. (see Example 1 in U.S. 62/911,096). For example, OR11A1 agonists all share a common earthy tonality even as they elicit an overall set of distinct tonalities (see Example 2 in U.S. 62/911,096). A volatile molecule may have greater than one tonality. For example, beta ionone has violet and blonde woody tonalities arising from the activation of OR5A1 and OR7A17, respectively (see Example 3 in U.S. 62/911,096).

As used herein, the terms “means of inputting” is, for example, a keyboard, mouse and/or touchscreen adapted to interact with a computing system in such a way to collect user input. In variants, the means of inputting are logical in nature, such as a network port of a computing system configured to receive an input command transmitted electronically. Such an input means may be associated to a GUI (Graphic User Interface) shown to a user or an API (Application programming interface). In other variants, the means of inputting may be a sensor configured to measure a specified physical parameter relevant for the intended use case.

As used herein, the terms “computing system” or “computer system” designate any electronic calculation device, whether unitary or distributed, capable of receiving numerical inputs and providing numerical outputs by and to any sort of interface, digital and/or analog. Typically, a computing system designates either a computer executing a software having access to data storage or a client-server architecture wherein the data and/or calculation is performed at the server side while the client side acts as an interface.

As used herein, the terms “digital identifier” refer to any computerized identifier, such as one used in a computer database, representing a physical object, such as a flavoring ingredient. A digital identifier may refer to a label representative of the name, chemical structure, or internal reference of the flavoring ingredient.

In the present description, the term “materialized” is intended as existing outside of the digital environment of the present invention. “Materialized” may mean, for example, readily found in nature or synthesized in a laboratory or chemical plant. In any event, a materialized composition presents a tangible reality. The terms “to be compounded” or “compounding” refer to the act of materialization of a composition, whether via extraction and assembly of ingredients or via synthetization and assembly of ingredients.

The embodiments disclosed below are presented in a general manner.

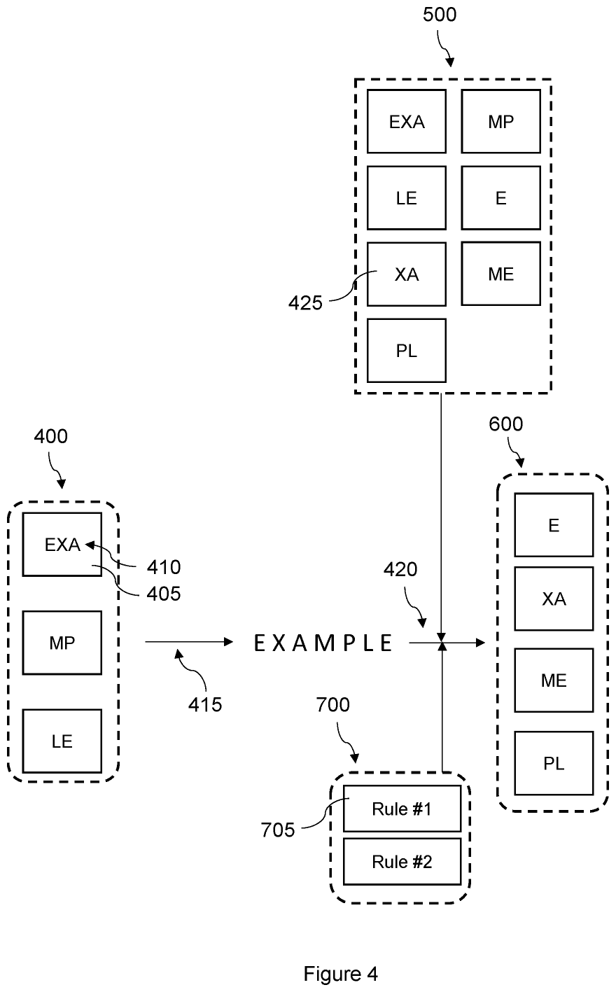

FIG. 4 shows, schematically, the concept at the basis of the present invention.

This figure represents:
an initially defined or input composition 400 of three ingredients 405, each ingredient being composed by several molecules 410; for example, a first ingredient is composed of molecules “E”, “X”, and “A”,
a step 415 of decomposition of the initial composition 400 into constitutive molecules of said composition 400, thus removing the consideration for the initially input ingredients; for example, the initial composition is decomposed into molecules “E”, “X”, “A”, “M”, “P”, “L”, “E”,
a database 500 of ingredients 425, including the initially selected ingredients to form the initial composition 400, each ingredient being associated with at least one constitutive molecule,
a rules definition module 700, which defines rules 705 for the reformulated composition 600, a rule associating a target value for a property for the target composition, ingredients and/or molecules,
a step 420 of reformulating the initial composition by selecting from the database 500, ingredients 425 to obtain at least the same constitutive molecules than the initially defined composition 400, the step 420 of reformulating enforcing the rules of the rules definition module 700 and using the database 500 of ingredients, and
the alternative composition 600, composed of ingredients both initially selected and subsequently selected during the step 420 of reformulating.

Molecules may further be associated with psychophysical properties which can correspond to reformulation rules (i.e., it is expected of the target composition 600 to provide a similar taste experience for users than the initially input composition 400).

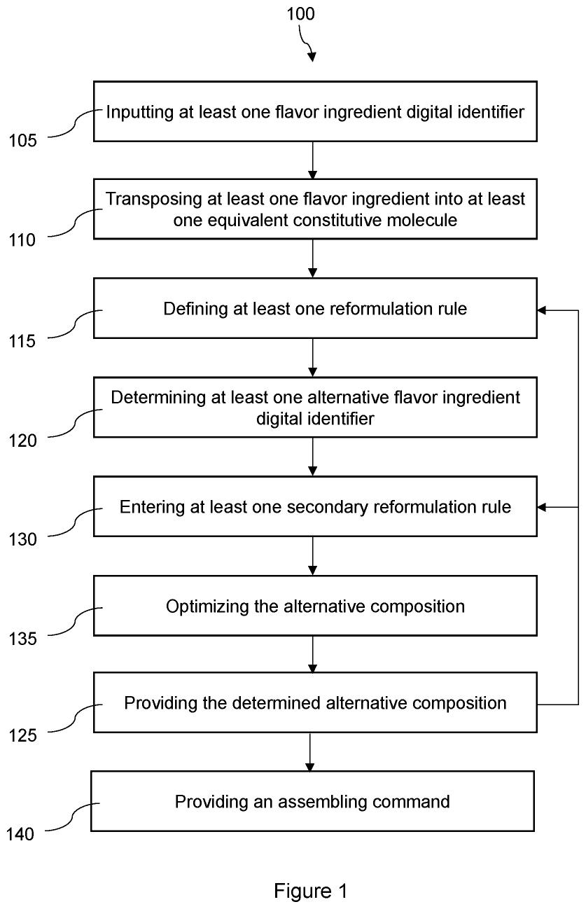

FIG. 1 shows a particular succession of steps of the method 100 object of the present invention. This computer-implemented method 100 to provide alternative flavor compositions comprises:
a step 105 of inputting, upon a computer interface, at least one flavor ingredient digital identifier, the resulting input corresponding to a composition of flavor ingredients, each ingredient digital identifier being associated with at least one equivalent constitutive molecule, each equivalent constitutive molecule being associated with at least one property,
a step 110 of transposing, by a computing system, the input composition into a list of at least one equivalent constitutive molecule as a function of at least one input ingredient digital identifier,
a step 115 of defining, upon a computer interface, at least one reformulation rule to be applied to the at least one transposed equivalent constitutive molecule and/or to at least one alternative flavor ingredient obtained during a step of determining,
the step 120 of determining, by a computing system, a selection of at least one alternative flavor ingredient represented by at least one alternative flavor ingredient digital identifier as a function of at least one reformulation rule, the resulting selection corresponding to an alternative composition of flavor ingredients, and
a step 125 of providing, upon a computer interface, the determined alternative composition of flavor ingredients.

The step 105 of inputting may be performed, via a computer interface, such as an API or any other digital input system. This step 105 of inputting may be initiated manually or automatically, via any means of inputting known to a Person Skilled in the Art. During this step 105 of inputting, at least one flavor ingredient digital identifier is selected to form a flavoring composition or formula. Such an identifier may be a commercial name, or a reference, for a material flavoring ingredient.

In particular embodiments, such as the one shown in FIG. 3, a user selects, upon a GUI associated with a computing system, at least one flavor ingredient digital identifier 305 in a list of available flavor ingredient digital identifiers. This selected flavor ingredient digital identifier is preferably associated with a relative or absolute quantity of said flavor ingredient in the composition.

The step 110 of transposing is performed, for example, by a computer software executed upon a computing device. During this step 110 of transposing, for example, a database linking flavor ingredient digital identifiers to corresponding equivalent constitutive molecules is used. Such a database may comprise, for example, an identifier for a flavor ingredient and, for each such identifier, at least one identifier for an equivalent constitutive molecule. For example, a flavor ingredient reference called “code 239” may be associated with identifiers such as “limonene”, “1-dodecanol”, “myrcene” and other such equivalent molecules.

At the end of this step 110 of transposing, the initial composition is exploded into constitutive molecules. It is worth considering that a single molecule may be associated with several ingredients, such that this relationship is not exclusive. This means, for example, that a total quantity of a particular equivalent constitutive molecule can be calculated as the sum of equivalent constitutive molecule quantity for all flavor ingredients comprising said molecule in the composition.

The step 115 of defining may be performed, via a computer interface, such as an API or any other digital input system. This step 105 of inputting may be initiated manually or automatically, via any means of inputting known to a Person Skilled in the Art. In a particular example, such as shown in FIG. 3, a GUI allows for a user to add, modify, or remove reformulation rules 310.

In particular embodiments, at least one reformulation rule is representative of:
an origin of at least one flavor ingredient, which can be, for example, naturally sourced or nature-identical, such as defined by the legislation of a particular country,
a geographical region of origin or destination of at least one molecule or ingredient, each molecule or ingredient being associated with at least one property representative of a geographical region of origin of said molecule or ingredient,
a geographical region of use for the composition, each equivalent constitutive molecule or ingredient being associated with at least one property representative of a use for the composition comprising said equivalent constitutive molecule or ingredient,
a composition production cost, each ingredient molecule being associated with at least one property representative of a production cost of said ingredient,
an ingredient regulatory status of at least one ingredient, each ingredient being associated with at least one property representative of an ingredient regulatory status of said ingredient, such regulatory status being optionally associated with at least one country in which the regulatory status is applicable,
an ingredient production ecological impact status of at least one ingredient, each ingredient being associated with at least one property representative of an ingredient production ecological impact of said ingredient,
an ingredient availability of at least one ingredient, each ingredient being associated with at least one property representative of an ingredient availability of said ingredient,
a Henry constant of at least one equivalent constitutive molecule, each equivalent constitutive molecule being associated with at least one property representative of a Henry constant of said equivalent constitutive molecule,
an odor detection threshold of at least one equivalent constitutive molecule, each equivalent constitutive molecule being associated with at least one property representative of an odor detection threshold of said equivalent constitutive molecule,
a maximum number of constitutive molecules or ingredients in the composition and/or
a dose response curve of at least one equivalent constitutive molecule, each equivalent constitutive molecule being associated with at least one property representative of a dose response curve of said equivalent constitutive molecule.

A dose response curve, in this context, refers to the logistic function relating psychophysical intensity perceived as a function of concentration of a particular molecule. A value representing said dose response curve may be an inflection point, a slope at inflection and/or a theoretical maximum intensity. Any other representative parameter, such as one disclosed in the publication “Multidimensional Visualization of Physical and Perceptual Data Leading to a Creative Approach in Fragrance Development,” by Christine Vuilleumier et al., in Perfumer Flavorist, volume 33 of September 2008 (pages 54 to 61).

A Henry constant value may be used to calculate the concentration of a particular molecule in the air as compared to the concentration in the flavor composition as a product of consumption.

A regulatory status value may be, for example, whether a molecule is kosher or halal.

An odor detection threshold corresponds to a concentration threshold at which the human nose is capable of detecting and/or recognizing the presence of the molecule.

Each said reformulation rule may take the form of a numerical value, in absolute or relative terms, and a comparator, such as “more than,” “equal to” or “less than” or the form of a label and a comparator, such as “is not” or “is.” In the latter case, an example of such a label is, for example, that an ingredient is or is not available for a specific reason due to regulatory reasons.

The step 120 of determining is performed, for example, by a computer software executed upon a computing device. During this step 120 of determining, for example, a linear combination of flavoring ingredients digital identifiers, in which:
each ingredient, or constitutive molecules if applicable, fits at least one, and preferably all, reformulation rules defined,
the composition of ingredients fits at least one, and preferably all, reformulation rules defined.

This linear combination can be performed according to any linear combination algorithm known to a Person Skilled in the Art, such as an Euclidean vector linear combination.

Such an algorithm is preferably suited for convex optimization.

At the output of this step 120 of determining, all or part of the flavor ingredients present in the initial composition may have been removed and/or the concentration and/or quantity of said flavor ingredients in the alternative composition may have been reduced or increased. Other flavor ingredients, not associated with the initial composition, may be added to the alternative composition.

The step 125 of providing is performed, for example, by displaying upon a computer screen the alternative composition and/or the initial composition in the shape of a radar chart for example. An example of such a step 125 of providing is shown, for example, in FIG. 3, in which an alternative 315 composition is provided based upon the input composition 305 and the reformulation rules 310 defined.

In particular embodiments, the method 100 object of the present invention comprises:
a step 130 of entering, upon a computer interface, at least one secondary reformulation rule to be applied on the alternative composition of flavor ingredients determined,
a step 135 of optimizing, by a computer system, the alternative composition of flavor ingredients determined as a function of the entered at least one secondary reformulation rule.

The step 130 of entering is performed, for example, similarly to the step 115 of defining.

The step 135 of optimizing is performed, similarly to the step of 120 determining. During this step 135 of optimizing, ingredients of the alternative composition are compared to the entered secondary reformulation rules and, if the secondary reformulation rules are not met, replaced by other ingredients.

In particular embodiments, each equivalent constitutive molecule is associated with at least one olfactory property, the step 120 of determining being further performed as a function of at least one said olfactory property.

Such an olfactory property may be, for example, a scent tonality of the molecule, a scent descriptor associated with said molecule or a psychophysical perceived intensity for the scent of the molecule. Such an olfactory property may be, for example, that the perceived intensity is below the detection threshold, i.e., close to zero. Such a system may also be applied to non-volatile molecules.

In particular embodiments, the method 100 object of the present invention comprises a step 140 of providing, to an assembling device, an assembling command of the alternative composition of flavor ingredients.

The assembling device is configured to materialize the composition during a step of assembling. Such a step of assembling may be performed in a variety of ways, such as in a laboratory or a chemical plant for example.

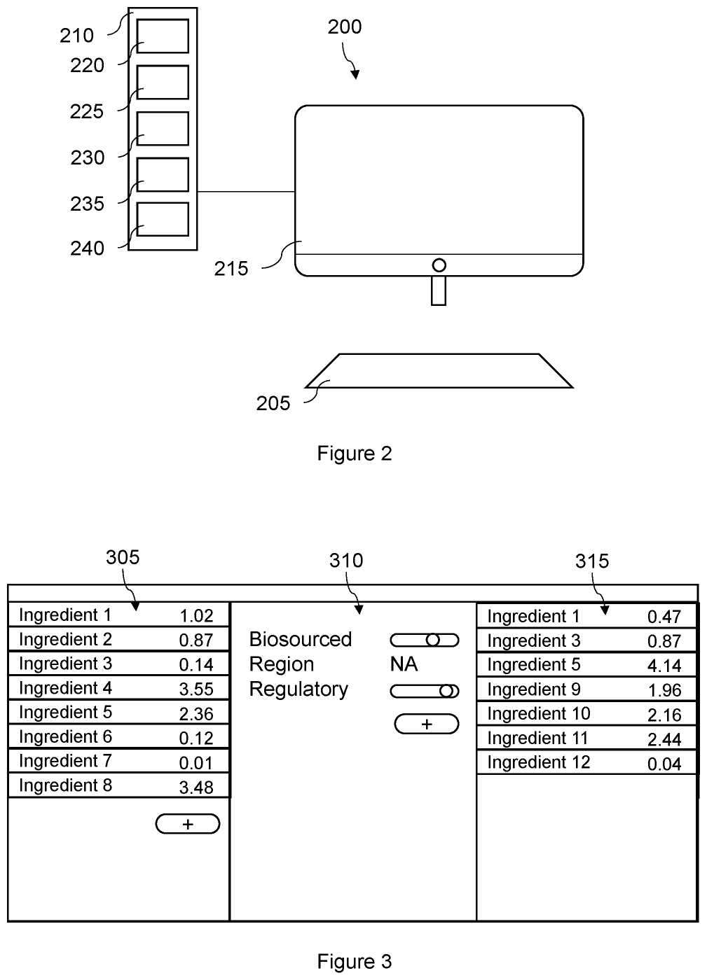

FIG. 2 represents, schematically, a particular embodiment of the system 200 object of the present invention. This system 200 to provide alternative flavor compositions, comprises:
means 220 of inputting, upon a computer 205 interface, at least one flavor ingredient digital identifier, the resulting input corresponding to a composition of flavor ingredients, each ingredient digital identifier being associated with at least one equivalent constitutive molecule, each equivalent constitutive molecule being associated with at least one property,
means 225 of transposing, by a computing 210 system, the input composition into a list of at least one equivalent constitutive molecule as a function of at least one input ingredient digital identifier,
means 230 of defining, upon a computer interface, at least one reformulation rule to be applied to the at least one transposed equivalent constitutive molecule and/or to at least one alternative flavor ingredient obtained by means of determining,
said means 235 of determining, by a computing system, a selection of at least one alternative flavor represented by at least one alternative flavor ingredient digital identifier as a function of at least one reformulation rule, the resulting selection corresponding to an alternative composition of flavor ingredients, and
means 240 of providing, upon a computer 215 interface, the determined alternative composition of flavor ingredients.

Particular embodiments of the means of the system 200 object of the present invention are disclosed in regards of FIGS. 1 and 3.

1. Computer-implemented method to provide alternative flavor compositions, comprising:
a step of inputting, upon a computer interface, at least one flavor ingredient digital identifier, the resulting input corresponding to a composition of flavor ingredients, each ingredient digital identifier being associated with at least one equivalent constitutive molecule, each equivalent constitutive molecule being associated with at least one property,
a step of transposing, by a computing system, the input composition into a list of at least one equivalent constitutive molecule as a function of at least one input ingredient digital identifier,
a step of defining, upon a computer interface, at least one reformulation rule to be applied to the at least one transposed equivalent constitutive molecule and/or to at least one alternative flavor ingredient obtained during a step of determining,
the step of determining, by a computing system, a selection of at least one alternative flavor ingredient represented by at least one alternative flavor ingredient digital identifier as a function of at least one reformulation rule, the resulting selection corresponding to an alternative composition of flavor ingredients, and
a step of providing, upon a computer interface, the determined alternative composition of flavor ingredients.

2. Method according to claim 1, which comprises:
a step of entering, upon a computer interface, at least one secondary reformulation rule to be applied on the alternative composition of flavor ingredients determined,
a step of optimizing, by a computer system, the alternative composition of flavor ingredients determined as a function of the entered at least one secondary reformulation rule.

3. Method according to claim 1, in which at least one reformulation rule is representative of a geographical region of origin of at least one ingredient, each ingredient being associated with at least one property representative of a geographical region of origin of said ingredient.

4. Method according to claim 1, in which at least one reformulation rule is representative of a molecule production ecological impact status of at least one equivalent constitutive molecule, each equivalent constitutive molecule being associated with at least one property representative of a molecule production ecological impact of said equivalent constitutive molecule.

5. Method according to claim 1, in which at least one reformulation rule is representative of a molecule availability of at least one equivalent constitutive molecule, each equivalent constitutive molecule being associated with at least one property representative of a molecule availability of said equivalent constitutive molecule.

6. Method according to claim 1, in which at least one reformulation rule is representative of a Henry constant of at least one equivalent constitutive molecule, each equivalent constitutive molecule being associated with at least one property representative of a Henry constant of said equivalent constitutive molecule.

7. Method according to claim 1, in which at least one reformulation rule is representative of an odor detection threshold of at least one equivalent constitutive molecule, each equivalent constitutive molecule being associated with at least one property representative of an odor detection threshold of said equivalent constitutive molecule.

8. Method according to claim 1, in which at least one reformulation rule is representative of a dose response curve of at least one equivalent constitutive molecule, each equivalent constitutive molecule being associated with at least one property representative of a dose response curve of said equivalent constitutive molecule.

9. Method according to claim 1, in which each equivalent constitutive molecule is associated with at least one olfactory property, the step of determining being further performed as a function of at least one said olfactory property.

10. Method according to claim 1, which comprises a step of providing, to an assembling device, an assembling command of the alternative composition of flavor ingredients.

11. System to provide alternative flavor compositions, comprising:
means of inputting, upon a computer interface, at least one flavor ingredient digital identifier, the resulting input corresponding to a composition of flavor ingredients, each ingredient digital identifier being associated with at least one equivalent constitutive molecule, each equivalent constitutive molecule being associated with at least one property,
means of transposing, by a computing system, the input composition into a list of at least one equivalent constitutive molecule as a function of at least one input ingredient digital identifier,
means of defining, upon a computer interface, at least one reformulation rule to be applied to the at least one transposed equivalent constitutive molecule and/or to at least one alternative flavor ingredient obtained by means of determining,
said means of determining, by a computing system, a selection of at least one alternative flavor ingredient represented by at least one alternative flavor ingredient digital identifier as a function of at least one reformulation rule, the resulting selection corresponding to an alternative composition of flavor ingredients, and
means of providing, upon a computer interface, the determined alternative composition of flavor ingredients.
